(19) United States
(12) Patent Application Publication
(10) Pub. No.: US 2018/0214650 A1
(43) Pub. Date: Aug. 2, 2018

(54) Oral Appliance for Use with Continuous Positive Airway Pressure Machines

(71) Applicant: John George Pender, Fairbanks, AK (US)

(72) Inventor: John George Pender, Fairbanks, AK (US)

(21) Appl. No.: 15/418,747

(22) Filed: Jan. 29, 2017

(51) Int. Cl. A61M 16/04; A61F 5/56; A61M 16/06

(52) U.S. Cl. CPC A61M 16/049; A61F 5/566; A61M 16/0627; A61M 16/0666; A61M 16/0605; A61F 5/56; A61M 16/0488; A61M 16/06; A61M 16/0683

(57) ABSTRACT

An oral appliance for users of continuous positive airway pressure (CPAP) machines. CPAP users who use a nose-only mask experience a flow of pressurized air out the mouth if the mouth is open. This causes the mouth to dry out and allows the return of obstructive sleep apnea. The oral appliance forms a seal in the user's mouth that prevents the flow of air out of the mouth, which restores full function to the CPAP machine.

### CROSS REFERENCE TO RELATED APPLICATIONS

Not Applicable

### STATEMENT REGARDING FEDERALLY SPONSORED RESEARCH AND DEVELOPMENT

Not Applicable

### BACKGROUND OF THE INVENTION

### 1. Field of the Invention

This invention relates to the field of accessories for continuous positive airway pressure (CPAP) machines and particularly to fitted mouthpiece accessories for CPAP machines.

### 2. Description of the Prior Art

Obstructive sleep apnea (OSA) is a serious sleep disorder caused by a blockage of the airway, usually when soft tissue in the throat sags during sleep. If left untreated OSA can result in a variety of health problems, including high blood pressure, stroke, heart failure, diabetes, depression and headaches. The headaches are caused by very low blood oxygen levels, which should fall no lower than 95%. OSA can reduce these levels to less—sometimes much less—than 80%, producing symptoms very much like carbon monoxide poisoning.

Mild cases of OSA may be treated by losing weight, avoiding alcohol and sleeping on ones side. Some patients report relief when they use an oral appliance that keeps their chin in position while asleep. Surgery may be required in some cases. There is currently no clinically effective drug therapy.

Patients with moderate-to-severe OSA are typically treated with CPAP machines. CPAP machines have been used to treat sleep apnea since the 1980s. An air pump pressurizes a hose which is connected to a mask over the patient's face. The pressure keeps tissue in the throat from sagging to the point where the airway is blocked. Many modifications have been made to the original apparatus over the last several decades to reduce noise, improve comfort, heat and/or humidify the air, incorporate anti-asphyxiation valves, etc. See, for example, U.S. Pat. Nos. 4,655,213, 5,065,765, 5,301,689, 5,567,127, 5,649,533, 6,123,071, 6,182,657, 6,561,190, 6,581,594, 6,595,212, 6,662,803, 6,823,865, 7,063,086, 7,159,587, 7,341,060, 7,527,055, 7,856,979, 7,926,487, 8,122,884, 8,337,145, 8,371,300, 8,839,791, 8,844,529, and 9,072,855.

It matters whether the patient sleeps with mouth open, even if infrequently. If the patient is wearing a nose-only mask and the lips are parted for any reason then the CPAP machine drives a continuous flow of air into the nose and out of the mouth. Once started, the flow of air works to keep the lips parted, especially at higher CPAP operating pressures. In and of itself this is not uncomfortable, but the flow of air causes two problems. First, the airway is no longer pressurized so the apnea returns, defeating the purpose of using a CPAP machine. Second, the flow of air will desiccate the mouth and/or nasal passages which is uncomfortable and eventually wakes the patient up. In the context of CPAP usage, “dry mouth syndrome” refers specifically to this situation, though it should be noted that the phrase can also refer to a medical condition called xerostomia which is unrelated to sleep apnea.

There are at present several strategies for avoiding CPAP dry mouth syndrome. There are medications (e. g. XYLIMELTS, which contain xylitol) and salves/lubricants (e. g. BIOTENE) that can help with the symptoms of dry mouth but they are not always effective and do nothing to relieve the effects of renewed sleep apnea. Most CPAP machines can be fitted with a humidifier; the humid air slows down the drying process but does nothing to relieve the effects of renewed OSA. The patient can keep the jaws clamped together with a chin strap and hope that this also keeps the lips from parting. As a last resort, patients can (and do) tape over the mouth every night and then remove the tape in the morning.

### BRIEF DESCRIPTION OF THE INVENTION

The invention is a simple oral appliance that blocks the flow of pressurized air out of the mouth. The invention improves the effectiveness of CPAP machines by maintaining airway pressure when the lips are parted. The invention consists of a thin, fitted strip made from a soft, flexible and impermeable material that sits in front of the teeth but behind the lips. The invention acts like a gasket around the perimeter of the mouth that blocks the flow of air even if the lips are parted.

It is an object of the invention to produce an oral appliance that blocks the flow of pressurized air out of the mouth that is made of a material that is sufficiently flexible to provide maximum comfort for the user.

It is another object of the invention to produce an oral appliance that blocks the flow of pressurized air out of the mouth that made from a material that has a pleasant taste, or no taste at all.

It is yet another object of the invention to produce an oral appliance that blocks the flow of pressurized air out of the mouth that made from a material that will not leach chemicals into the patient's system or degrade in any way from prolonged contact with saliva.

It is yet another object of the invention to produce an oral appliance that blocks the flow of pressurized air out of the mouth that is not so thin and flexible that the patient will aspirate or swallow it while asleep.

It is yet another object of the invention to produce an oral appliance that blocks the flow of pressurized air out of the mouth that is made from a material durable enough that it will not be substantially damaged if the patient chews on it while asleep.

### BRIEF DESCRIPTION OF THE DRAWINGS

FIG. 1 is a front perspective view of a first embodiment of the invention.

FIG. 2 is a front detail view of the invention as positioned in a user's mouth.

FIG. 3 is a side detail view of the invention as positioned in a user's mouth.

FIG. 4 is a top perspective view of a second embodiment of the invention.

FIG. 5 is a side perspective view of the second embodiment of the invention.

FIG. 6 is a top perspective view of a third embodiment of the invention.

FIG. 7 is a side perspective view of the third embodiment of the invention.

### DETAILED DESCRIPTION OF THE INVENTION

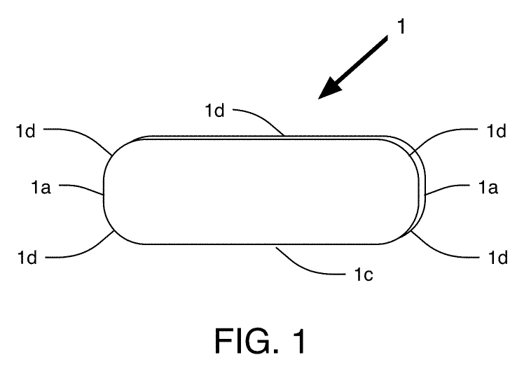

FIG. 1 shows a first embodiment of the invention 1, consisting of a thin strip of food-grade material (e. g. NSF 51 silicone rubber) which is very soft, flexible, durable and tasteless. The preferred thickness of this strip, for maximum patient comfort and safety is 1 mm. The strip is generally rectangular in shape and has side walls 1a, a bottom 1c and a top 1d as shown. The overall dimensions are chosen to make sure the invention works well as a gasket against the inside of the lips. In the preferred embodiment, this width is somewhat wider than the opening of the mouth, approximately 11-12 cm for an adult male. The height is somewhat less than the distance between the top of the lip in the upper jaw and the bottom of the lip in the lower jaw, approximately 3-4 cm for an adult male. Obviously, these dimensions are readily adjusted for smaller or larger mouths as needed. This embodiment can also be made of a rigid, food-grade material which is molded to fit in place.

The corners 1d may be rounded to make the invention more comfortable. When the patient is ready to sleep with the CPAP machine, the user simply places the device into his or her mouth in front of the teeth and behind the lips.

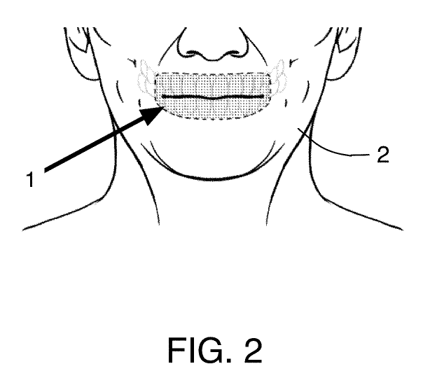

FIG. 2 shows a front view of the invention 1 in place in the mouth of a user 2 (dashed line and shading).

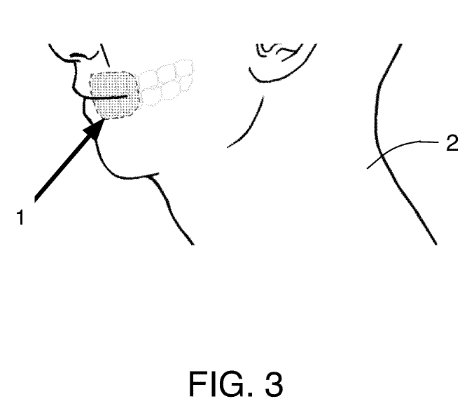

FIG. 3 is a side view of the invention 1 (dashed line and shading) in the mouth of a user 2.

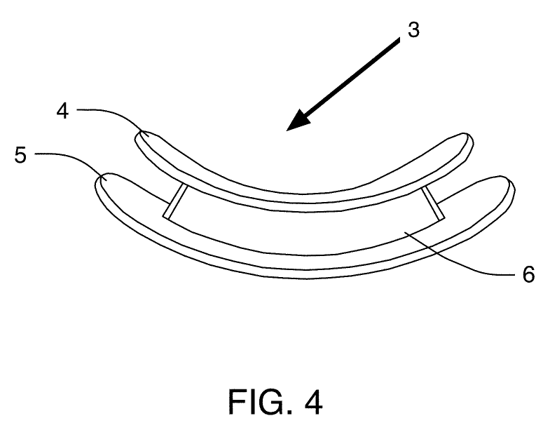

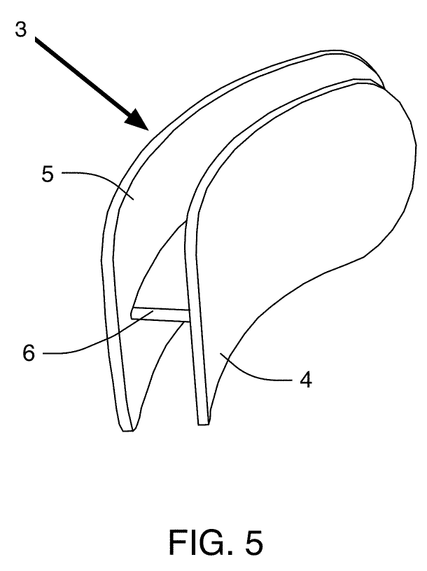

One problem often experienced is that if the CPAP operating pressure is high it is more difficult to keep the lips sealed. FIG. 4 is a top perspective view of a second embodiment 3 of the invention. An inner flange 4 goes between the lips and the teeth and forms a seal against the inner surface of the lips. An outer flange 5 goes over the outside of the mouth and forms a seal against the outer surface of the lips. The inner flange and the outer flange need not have the same lateral dimensions. The inner flange and the outer flange are joined by a single tab 6 that passes between the lips. FIG. 5 is a side perspective view of the second embodiment of the invention 3. As in the case of the first embodiment, this embodiment is also made of food-grade material (e. g. NSF 51 silicone rubber) which is very soft, flexible, durable and tasteless. Here too, the invention can be made from a rigid, food-grade material which is molded to fit in place.

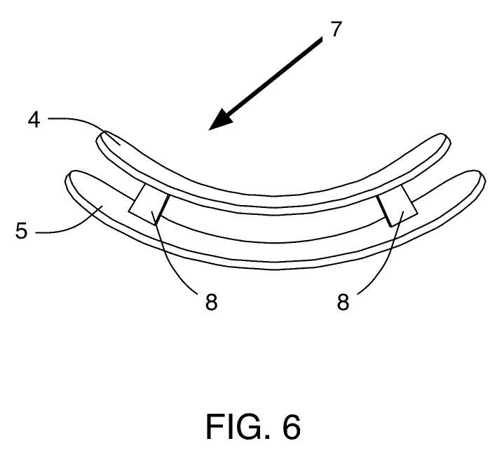

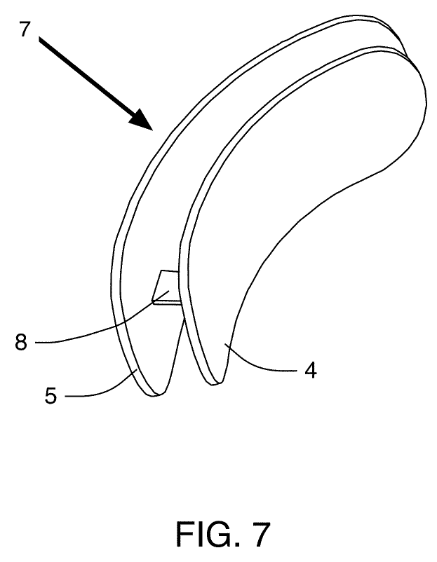

FIG. 6 is a top perspective view of a third embodiment 7 of the invention. FIG. 7 is a side perspective view of of the third embodiment of the invention. In this embodiment, a pair of tabs 8 join the inner flange 4 to the outer flange 5. The tabs are located near the corners of the mouth. Again, this embodiment is also made of food-grade material (e. g. NSF 51 silicone rubber) which is very soft, flexible, durable and tasteless. As before, the invention can also be made from a rigid, food-grade material which is molded to fit in place.

Full-face and nose-only CPAP masks are often fitted with an anti-asphyxia valve (AAV), usually incorporated into the elbow fitting which connects the mask to the hose. See, for example, U.S. Pat. Nos. 8,517,024 and 9,295,805. Under normal operating conditions the air pressure inside the mask is higher than the air pressure outside the mask and this keeps the AAV closed. In the event of a power outage the air pressure in the mask falls to ambient which allows the AAV to open so that the patient can more easily breathe fresh air. Inasmuch as the purpose of this invention is to block the flow of air through the mouth, it is recommended that patients use a CPAP mask fitted with an AAV when using this invention.

The present disclosure should not be construed in any limited sense other than that limited by the scope of the claims having regard to the teachings herein and the prior art being apparent with the preferred form of the invention disclosed herein and which reveals details of structure of a preferred form necessary for a better understanding of the invention and may be subject to change by skilled persons within the scope of the invention without departing from the concept thereof.

I claim:

1. An oral appliance for use with continuous positive airway pressure machines comprising a generally rectangular strip of food-grade material, being soft, flexible, and durable said generally rectangular strip being sized to fit in a user's mouth between a user's teeth and lips.

2. The oral appliance of claim 1 wherein the generally rectangular strip has a thickness of about 1 mm.

3. The oral appliance of claim 1 wherein the generally rectangular strip has four rounded corners.

4. The oral appliance of claim 1 wherein the generally rectangular strip has has a width of about 11-12 cm.

5. The oral appliance of claim 1 wherein the generally rectangular strip has a height of about 3-4 cm.

6. The oral appliance of claim 1 wherein the food grade material is a food-grade silicone rubber, being soft, flexible, durable and tasteless.

7. The oral appliance of claim 1 wherein the food grade material is rigid, and molded to fit in place.

8. An oral appliance for use with continuous positive airway pressure machines comprising:
a) an inner flange, made of a food grade material, that is placed between a user's lips and the teeth;
b) an outer flange, made of a food grade material that is placed over the outside of said user's mouth; and
c) one or more connecting tabs, made of a food grade material, attached to said inner and outer flanges.

9. The oral appliance of claim 8 wherein the inner flange forms a seal against the inner surface of the user's lips.

10. The oral appliance of claim 8 wherein the outer flange forms a seal against the outer surface of the user's lips.

11. The oral appliance of claim 8 wherein the food grade material is a food-grade silicone rubber, being soft, flexible, durable and tasteless.

12. The oral appliance of claim 8 wherein the food grade material is rigid, and molded to fit in place.

13. A method of sealing a user's mouth when using a continuous positive air pressure machine by using a oral appliance comprising the step of, prior to sleep, placing a generally rectangular strip between the user's teeth and lips.

14. The method of claim 13 wherein the generally rectangular strip has a thickness of about 1 mm.

15. The method of claim 13 wherein the generally rectangular strip has four rounded corners.

16. The method of claim 13 wherein the generally rectangular strip has has a width of about 11-12 cm.

17. The method of claim 13 wherein the generally rectangular strip has a height of about 3-4 cm.

18. The method of claim 13 wherein the food grade material is a food-grade silicone rubber, being soft, flexible, durable and tasteless.

19. A method of sealing a user's mouth when using a continuous positive air pressure machine by using a oral appliance comprising the step of prior to sleep, placing a device having an inner flange an outer flange, and one or more connecting tabs made of food grade material, in the user's mouth such that the inner flange is positioned between a user's lips and the teeth; and the outer flange is positioned over the outside of said user's mouth.

20. The method of claim 19 wherein the inner flange forms a seal against the inner surface of the user's lips.

21. The method of claim 19 wherein the outer flange forms a seal against the outer surface of the user's lips.

22. The method of claim 19 wherein the food grade material is a food-grade silicone rubber, being soft, flexible, durable and tasteless.
